(19) United States
(12) Patent Application Publication
(10) Pub. No.: US 2019/0269891 A1
(43) Pub. Date: Sep. 5, 2019

(54) URETERAL GUIDEWIRE AND STENT

(71) Applicant: Lawrence E Lykins, Gainesville, GA (US)

(72) Inventor: Lawrence E Lykins, Gainesville, GA (US)

(21) Appl. No.: 16/269,455

(22) Filed: Feb. 6, 2019

Related U.S. Application Data
(60) Provisional application No. 62/709,921, filed on Feb. 6, 2018.

(51) Int. Cl. A61M 25/09; A61M 27/00; A61F 2/04; A61F 2/95

(52) U.S. Cl. CPC A61M 25/09; A61M 27/008; A61F 2002/048; A61F 2/95; A61M 2205/584; A61F 2/04

(57) ABSTRACT

A guidewire for use with ureteral stents has an elongated shape with a proximal end and a distal end. The guidewire has indicia along the proximal end to indicate the length of the guidewire that was introduced into the patient. Based on the indicia or the visual inspection, a physician can select the appropriately sized stent to place in the patient.

### CROSS-REFERENCE TO RELATED CASE

This application claims priority to and the benefit of the U.S. provisional patent application Ser. No. 62/709,921, filed on Feb. 6, 2017, the entirety of which is incorporated herein by reference.

### TECHNICAL FIELD

This disclosure generally relates to urinary catheters and stents. More particularly, this disclosure relates to guidewires having varying indicium (e.g., mark size markings) along their length.

### BACKGROUND

Insertion of J stent is a very common procedure performed by urologists and physicians. This procedure is often performed to relieve ureteral obstruction due to stones, tumors, ureteral kinks, or stricture on an urgent basis. Ureteral stents are small tubes inserted into the ureter to treat or prevent a blockage that prevents the flow of urine from the kidney to the bladder. An increasing number of urologists or physicians are using a flexible cystoscope and local anesthetic for J-stent insertion. In most cases, the procedure is straightforward. After placement of guidewire, the physician pushes the ureteral stent upwardly on the wire until the physician no longer is able to grip the lowermost or distal end of the tube body.

Urinary stent typically have a bladder curl made from a soft material to prevent migration and irritation. If the stent is placed up too far or not far enough, the patient experiences discomfort, which may result in physician return visits or/and replacement of the stent. Patient discomfort from these stents comes from a number of areas. One such area is that the bladder curl can rub and irritate the inside of the bladder, especially the sensitive trigone area. Another such area is that flank pain is perceived by the patient due to the reflux occurring as the bladder voids and urine is pushed up through the stent lumen back into the kidney.

When a physician places a stent in a ureter, he/she first passes a radiopaque guidewire up the ureter. When the floppy end of the guidewire reaches the kidney pelvis, it coils in the renal pelvis. This allows the physician to see this on fluoroscopy and know the guidewire is in the kidney pelvis. Presently, when a physician introduces a stent in a ureter over the guidewire, the urologist estimates the length of the stent that is suitable for the patient. This estimation is usually done by assessing the patient's height, which is only a partial predictor of the appropriately sized stent. For illustration, stents typically come in even numbered lengths, i.e., 20 cm, 22 cm, 24 cm, 26 cm, 28 cm, 30 cm—the physician selects the stent size based on the height of patient, which is probably a good predictor over half of the time. If the urologist places a stent that is too short, the stent retracts up into the ureter and a separate procedure may be required to get the stent out of the ureter. If the stent is too long, too much of the stent ends up in the bladder, which causes a lot of discomfort to the patient.

Accordingly, there is a need for a technique and device that can improve the selection of a stent or the size thereof suitable for a patient. It is to this need, among others, that this invention is directed.

### SUMMARY

This disclosure includes a guidewire for use with ureteral stents. The guidewire can have an elongated shape with a proximal end and a distal end. There can be a j-shaped curve at the distal end. The guidewire has indicia along the proximal end to indicate the length of the guidewire that was introduced into the patient. Based on the indicia or the visual inspection of the guidewire, the physician can select the appropriately sized stent to place in the patient.

One embodiment includes a method for inserting a ureteral stent into the ureter in a patient comprising (1) introducing into the ureter a guidewire having indicia at the proximal end of the guidewire into the patient, (2) examining the indicia with respect to the patient, (3) selecting a stent from a plurality of varying sized stents based on the amount of guidewire introduced into the patient as examined and as indicated by the indicia on the guidewire, and (4) advancing the selected stent into the patient. The indicia are preferably defined color segments, which are easily visible to a person.

Another embodiment includes a guidewire and stent system having a plurality of stents of varying sizes and a guidewire. The stents are elongated, relatively flexible, and tubular member having at least one drainage opening extending through a wall thereof. The guidewire has a proximal end and distal end and indicia on a proximal end to permit the proximal end to facilitate the placement of the stent member into the patient. The stent member receives the guidewire, and the stent member may be selected based on the indicia along the guidewire.

### FIGURES

FIG. 1 illustrates a guidewire having indicia thereon together with a stent,

FIG. 2 illustrates an exemplary guidewire with indicia,

FIG. 3 illustrates the guidewire placed into a patient in which the indicia is visible to the physician,

FIG. 4 is a schematic isometric view illustrating the ureteral stent in position between the kidney and the bladder.

### DETAILED DESCRIPTION

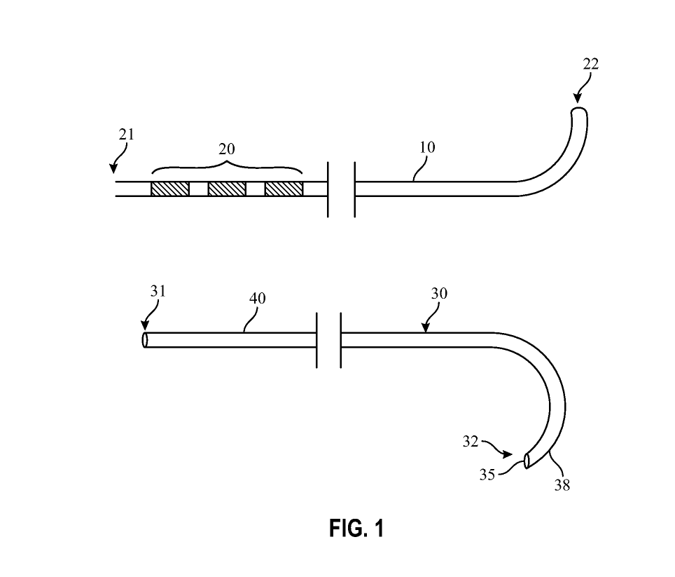

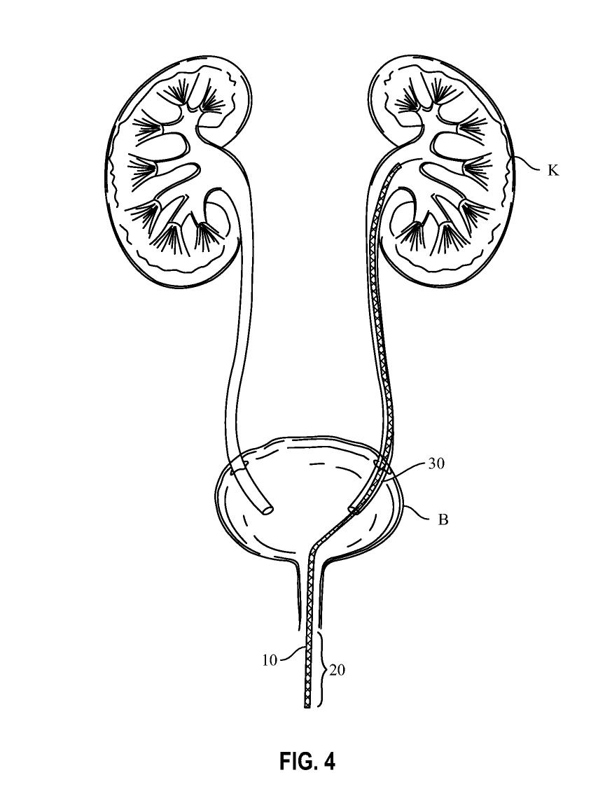

Ureteral stents are small tubes inserted into the ureter to treat or prevent a blockage that prevents the flow of urine from the kidney K to the bladder B. A stent is a small hollow tube that is placed into the ureter. During use, the ureteral stent is installed by a physician using an elongated guidewire 10 that extends from the physician's instrument known as a cystoscope, through the ureteral orifice, and then into the lumen of ureter until the guide wire reaches the patient's renal pelvis and kidney K, as shown in FIGS. 1 and 4. The physician inserts the uppermost or proximate end of the guidewire 10 and then selects the appropriately sized stent 30, based on the indicia 20, for use in the patient. When placed in the ureter, the top portion of the stent has a small curl (e.g., j-shaped curve) that sits in the kidney and the opposite ends curls in the bladder.

FIG. 1 illustrates one embodiment of the guidewire 10 having visible indicia 20 for determining the distance the guidewire 20 has been inserted into the ureter. The stent 30, currently available in an array of sizes (e.g., 22 cm, 24 cm, or 26 cm), comprises an elongated tubular member 35 having a relatively straight body portion 40 and opposed proximal end portion 31 and distal end portion 32. The distal end portion 32 is provided with a shape for retaining the distal end portion 38 in a kidney K and a shape for retaining the proximal end portion 31 in a bladder B. After or as the guidewire is placed into the patient, the physician can select the appropriate sized stent to place in the patient. The appropriate size will minimize patient discomfort and reduce the uncertainty for the physician.

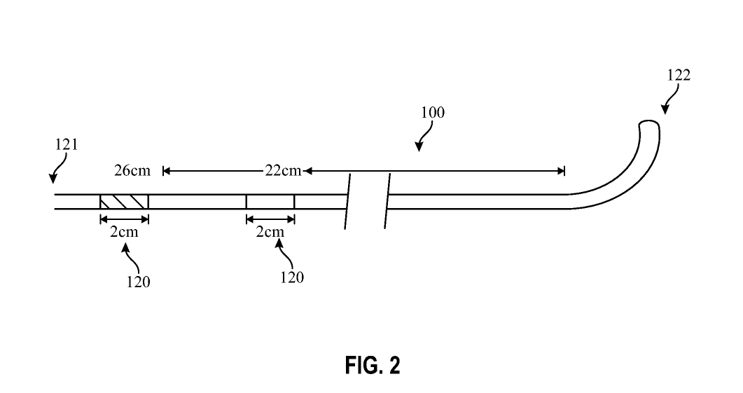

As can be seen from FIGS. 1 and 2, the guidewire 10 has indicia 20 at the proximal end 21 of the guidewire. The indicia 20 can indicate the amount or the length of the guidewire 10 that is introduced into the patient, and the appropriately sized stent 30 can be selected based on the length of the guidewire 10 in the patient. The indicia may be bands, colors, letters, numbers or any other indicator. The indicia 20 may be a scale to indicate the amount of the guidewire entering the patient. The indicia 20 may be etched markings on the guidewire 10. Preferably, the indicia are bold and visible to the physician. Preferably, the indicia is a band and greater than 1 cm or 2 cm.

In one example shown in FIG. 2, the guidewire 100 has a radiopaque hydrophilic nitinol wire that is at the about 0.32 or 0.35 inches in diameter and 26 cm long. The total length of the guidewire 100 is about 240 cm, and the distal end 122 may be floppy for 3 cm. In this example, the indicia 120 may be a white 2 cm band is on the wire at 22 cm from the proximal end 121 or tip of the floppy part of the guidewire 100. Other indicia 120 may include a bright green 2 cm band that is at a 26 cm mark on the wire. In one example, the markings are from before the j-curve to the proximal end 121. An urologist or physician can then use these bands 120 through visual inspection and can tell what length of stent will best fit the length of ureter or patient. From the indicia 120, the physician can select an appropriately sized stent for placement in the patient. Once the stent is properly positioned, the guidewire 100 can be removed leaving the stent 30 in place.

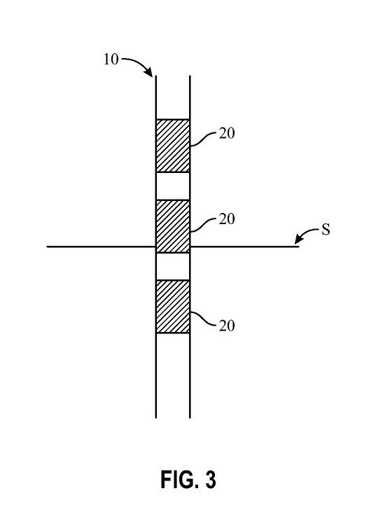

FIG. 3 illustrates an embodiment in which the guidewire 10 is introduced into a patient and in which a portion of the guidewire 10 is outside of the skin S of the patient for visual inspection. As can be seen, the amount of the guidewire 10 and indicia 20 visible to the physician are based on the size of the ureter of the patient. The physician then can select, based on professional judgment and the indicia, the appropriately sized stent 30 to place in the patient.

Another embodiment includes a simplified and more efficient catheterization/stent placement procedure. As is further described below, certain embodiments disclosed herein may be capable of achieving various advantages, including by one or more of the following: (1) providing a physician with a plurality of multiple sized stents or catheters for potentially placing into a patient; (2) introducing a guidewire with indicia into the patient to an desired position, (3) using the indicia to determine the length of stent that optimally fit the patient or the ureter, and (4) placing the selected sized stent onto the guidewire and ultimately in the patient. The physician selects the stent or the size of the stent based on the visual indicia on the guidewire. The stent is normally inserted in the ureter by placing the distal end of the stent onto the proximal end of the guidewire 10. Once the stent 30 is properly positioned, the guidewire 10 can be removed leaving the stent 30 in place.

In one embodiment, the physician has access to an array or varying sized stents 30. The ureteral stent 30 may have lengths varying from about 20 cm to about 30 cm and having outer diameters in the range of about 1 mm to about 4 mm. The outer diameters of the stents may range from about 1.5 mm to about 3 mm and most preferably the stent will have a wall thickness in the range of about 0.25 mm to about 1 mm. The stents 30 may made of a suitable flexible material which is soft and stiff enough for the intended purpose and which may have a radiopaque material. The stent may be supplied in various sizes and lengths. The physician selects the optimally sized stent based upon his/her professional judgment and the indicia visible on the guidewire after introduction into the patient. Ureteral stents are know those in the skill in the art and available for more than 40 years.

The ureteral stent may optionally be radiopaque. A radiopaque ureteral stent is capable of having its placement confirmed by X-ray or fluoroscopy. The polymers that compose the ureteral stent may be radiopaque, or the stent may include radiopaque markers. For example, a ureteral stent may include radiopaque markers at regular intervals along the length of the ureteral stent.

The ureteral stent may optionally include a coating on the external portion of the ureteral stent. A hydrophilic coating may be used to improve patient comfort or to facilitate insertion. An antibacterial or antimicrobial coating, such as heparin, may be used to reduce infection or encrustation. The ureteral stent may be coated with a single substance, or with a combination of multiple substances.

Specific embodiment generally relates to ureteral stents. By selecting the appropriately sized stent, the physician can reduce (compared with longer or shorter ureteral stents) patient discomfort when the stent is placed within the patient's body.

Methods for introducing a guidewire or stent into a patient are known to those of ordinary skill in the art. Various methods are used for inserting a ureteral stent into a patient. In one example, the ureteral stent is inserted though the urethral opening of a patient, advanced through the bladder and the ureter and subsequently placed in the kidney of the patient.

In certain embodiments of medical devices according to the invention, the stent may have, for example, a circular cross-section. The stent 10 may have a substantially constant cross-sectional area along its length. Alternatively, the stent 10 may be tapered from about the distal end portion to about the proximal end portion with the size of the section decreasing from about 7 French to about 3 French. Other French sizes and tapering configurations are useful depending upon either the average size in a population or the actual size in an individual of the body structure or structures into which the device is placed.

The stent 30 may be constructed from any of a number of materials. Those materials that are useful include, for example, materials that are able to flex but also retain their shape, to a degree, when they are perturbed. Additionally, useful materials are, for example, materials that have a resilient quality, being able to regain at least some of their original shape when the stent 30 ceases to be perturbed and/or resist, for example, compression.

The ureteral stent may be formed from a number of different biocompatible materials. The ureteral stent 30 may consist of one material or may be formed, for example by extrusion, of two or more materials along its length. For example, in one embodiment, the distal end portion of the ureteral stent 30 is formed from a first material having a first durometer and the proximal end portion is formed from a second material, which is softer and/or more flexible than the first material, having a second durometer different than the first durometer. Accordingly, the proximal end portion may be made of a softer or more flexible material than that of the distal end and/or the medial portion.

In an alternative embodiment, the indicia may be placed along the entire guidewire 10, so to assist the physician in determine amount of guidewire 10 being introduced into the patient.

Dimensions for the ureteral stent 10 of the present invention are not critical, however, they include internal diameters of 4.5 to 8.5 French, and lengths ranging from 20 cm. to 32 cm.

Identical concerns that are mentioned above with respect to stents are also applicable in the catheter arts, which include, without limitation: intravenous catheters, guiding catheters, sheaths, umbilical catheters, trocar catheters, heart catheters (including, valvostomy catheters, angioplasty catheters, arthroscopy catheters, and the like), perfusion catheters, suction catheters, oxygen catheters, endoscopy catheters, endotracheal tubes, stomach tubes, feeding tubes, lavage tubes, rectal tubes, urological tubes, irrigation tubes, aneurysm shunts, stenosis dialators, trocars, and inserters, generally.

While the invention has been described in connection with specific embodiments thereof, it will be understood that it is capable of further modification, and this application is intended to cover any variations, uses, or adaptations of the invention following, in general, the principles of the invention and including such departures from the present disclosure as come within known or customary practice in the art to which the invention pertains and as may be applied to the essential features hereinbefore set forth, and as fall within the scope of the invention and the limits of the appended claims.

1. A guidewire for receiving a stent for a patient, comprising a proximal and distal end and indicia on a proximal end to permit the proximal end to facilitate the placement of the stent member into the patient, wherein the stent member receives the guidewire and the indicia is visually seen an unaided eye.

2. The guidewire of claim 1, wherein the indicia includes multiple bands along the proximal end, wherein the bands are greater than 1 centimeter.

3. The guidewire of claim 1, wherein the bands are greater than 2 centimeters.

4. The guidewire of claim 1, wherein the guidewire is a metallic wire.

5. The guidewire of claim 1, wherein the indicia includes multiple colors.

6. The guidewire of claim 1, wherein a j-shaped curve is at the distal end.

7. The guidewire of claim 1, wherein the indicia is along the proximal end of the guidewire.

8. The guidewire of claim 1, wherein the indicia is a color.

9. A guidewire and stent system, comprising
a. a plurality of stents of varying sizes, wherein the stents are elongated, relatively flexible, and tubular member having at least one drainage opening extending through a wall thereof; and
b. a guidewire having a proximal and distal end and indicia on a proximal end to permit the proximal end to facilitate the placement of the stent member into the patient, wherein the stent member receives the guidewire.

10. The system of claim 9, wherein the indicia includes multiple bands along the proximal end, wherein the band is greater than 1 centimeter.

11. The system of claim 9, wherein the bands are greater than 2 centimeters.

12. The system of claim 9, wherein the stent is a ureter stent.

13. A method for inserting a ureteral stent into the ureter in a patient comprising:
introducing into the ureter a guidewire having indicia at the distal end of the guidewire into the patient,
examining the indicia with respect to the patient,
selecting a stent from a plurality of varying sized stents based on the amount of guidewire introduced into the patient as examined and as indicated by the indicia on the guidewire,
advancing the selected stent in the patient.

14. The method of claim 13, wherein the guidewire is introduced into the ureter of the patient and placed at a desired location between a bladder and a kidney.

15. The method of claim 13, wherein the size of the stent fits the patient based on the size of the ureter of the patient.

16. The method of claim 13, further comprising the step of removing the guidewire from the patient.

17. The method of claim 13, wherein the physician inspects the guidewire after introduction into the patient so to determine the desired length of the stent.

18. The method of claim 13, wherein the indicia is a color.
